(19) United States
(12) Patent Application Publication
(10) Pub. No.: US 2025/0368980 A1
(43) Pub. Date: Dec. 4, 2025

(54) METHOD AND KIT FOR MANUAL AND AUTOMATIC SAMPLE PREPARATION FOR LONG-READ SEQUENCING

(71) Applicant: IST Innuscreen GmbH, Berlin (DE)

(72) Inventors: Timo Hillebrand, Hoppegarten (DE); Elmara Graser, Berlin (DE); Kristin Wessel, Buergel OT Gniebsdorf (DE)

(73) Assignee: IST Innuscreen GmbH, Berlin (DE)

(21) Appl. No.: 18/876,717

(22) PCT Filed: Jun. 21, 2023
(86) PCT No.: PCT/EP2023/066800

§ 371 Date: Feb. 18, 2025

(30) Foreign Application Priority Data
Jun. 21, 2022 (DE) 10 2022 115 445.9

(51) Int. Cl. C12N 15/10

(52) U.S. Cl. CPC C12N 15/1006; C12N 15/1093

(57) ABSTRACT

A method and kit may extract high molecular weight DNA from a biological sample and/or purify a DNA library. In the method, after optional lysis of the biological sample and/or after preparation of the DNA library, the mixture is brought into contact with a combination of a polyether, preferably polyethylene glycol, and at least one salt, preferably magnesium chloride, in the absence of monohydric alcohols. Subsequently, the DNA is bound to a solid phase and then the DNA is purified without the use of monohydric alcohols. The method may be performed manually or automatically.

### STATE OF THE ART

New Generation Sequencing (NGS) technology is becoming increasingly important in the natural sciences, medicine and other related fields. The aim of comprehensive genome sequencing is not only to identify specific target sequences (pathogen detection, mutation detection, etc.), but also to elucidate the entire sequence of an organism. Logically, the length of the sequenced genome segments is the main focus of such questions using these new sequencing technologies (e.g. nanopore sequencing; Oxford Nanopore Technologies): the longer the sequencing reads, the easier and more resource-efficient is their bioinformatic analysis of the generated sequences and their assignment to the entire genome.

There are currently a number of sequencing platforms on the market with different abilities to capture longer DNA fragments. For example, the Nanopore sequencing platform is from Oxford Nanopore Technologies can be used to sequence fragments of megabase length (Ultra-Long DNA Sequencing Kit SQK-ULK001) without pre-amplification, provided that high-quality, high-molecular DNA is used.

But the theoretical possibility of sequencing longer fragments is not sufficient for the success of the entire sequencing process. The DNA sample should originally contain the required DNA fragment length and it should be possible to recover this DNA in the required quantity after tagmentation (production of a DNA library for subsequent nanopore sequencing). For the preparation of the DNA sample, Oxford Nanopore Technologies recommends the Monarch HMW DNA Extraction Kits (New England Biolabs). The Monarch HMW DNA Extraction Kits (New England Biolabs) are recommended by Oxford Nanopore Technologies for the preparation of the DNA sample. The total time required is estimated at 100 minutes in the protocol. But this method is not suitable for the reprocessing of DNA after tagmentation. The kits are based on the binding of high molecular DNA to glass beads. But the process requires alcoholic components (isopropanol and ethanol) both for binding the DNA and for the necessary washing steps. The problem is that so-called transposase fragmentation-based DNA libraries (DNA libraries) are produced in order to sequence the ultra-long DNA. After fragmentation of the DNA with transposases, accompanied by simultaneous incorporation of transposon sequences, the adaptors coupled to the so-called motor protein are attached. Once this DNA library has been generated, the unbound adaptors must be removed from the library so that they do not unnecessarily overload the sequencing pore capacities. This purification must mandatorily be carried out without isopropanol or ethanol etc. so that the motor protein is not destroyed. This means that it is not possible to use known classical purification methods, which are known to be highly efficient and fast (e.g. binding of DNA to solid phases by using ethanol or isopropanol and using isopropanol or ethanol-containing wash buffers).

For the purification of a DNA library for subsequent nanopore sequencing, ONP recommends carrying out a spermine precipitation (time required approx. 12 hours). For this purpose, it is recommended to prepare a 16.8 millimolar spermine solution and mix it 1:1 with the prepared DNA library. After a rotator-mixer incubation, the sample is centrifuged in order to generate the DNA pellet. The pellet should be dissolved in an elution buffer for at least 12 hours. The problem with this method lies in the ratio between the spermine and DNA concentration. The lower the DNA concentration, the higher the spermine concentration should be (E. Raspaud et al. Precipitation of DNA by Polyamines: A Polyelectrolyte is Behavior, Biophysical Journal 1998). This means that the slightest deviation of the specified DNA concentration leads to an inefficient precipitation reaction and thus to a loss of DNA. An increase in spermine concentration then again leads to the risk of potential “clogging” of the sequencing pores. In addition, resuspension of the pelleted DNA for more than 12 hours makes it impossible to prepare the experiment in one day.

Alternative variants for the purification of a DNA library for nanopore sequencing are described. A kit from Circulomics is commercially available (Matthew W. Mitchell et al High molecular weight DNA extraction and long-read next generation sequencing of human genomic reference standards. ASHG Virtual Meeting 2020). A Circulomix Nanobind disk is added to the prepared library and bound to the disk using an alcohol-free binding buffer, washed alcohol-free and then eluted. Here as well, you should wait at least 12 hours before the DNA can be used for sequencing. This kit is very expensive. Furthermore, it cannot be used for the previous extraction of high molecular weight DNA. This requires another kit. The process is also not automated.

Another published method for purification of the DNA library is based on the use of hexamminecobalt (III) chloride and glass beads (Inswasti Cahyani et al. Finding Nemo in One Day: Ultra-Long ONT Library Preparation from Cell to Flowcell in One Day, wwvw.protocols.io).

The prior art also includes the two German patent applications DE 10 2017 204 267 A1 and DE 10 2015 216 558 A1 as well as the corresponding patent applications WO2018/1 67138A1, EP3596215A1, US20200255820A1 resulting from it, the German patent no. DE102017204267B4 on the one hand and on the other hand the publications WO2016/169678A1 and the U.S. Pat. No. 10,934,540B2 and EP3286325A1. DE 10 2017 204 267 A1 describes the detachment of DNA bound to glass particles by using a washing solution. DE 10 2015 216 558 A1 discloses the use of a rough surface for binding nucleic acids.

But none of these listed methods provides a integral solution for sample preparation for nanopore sequencing (extraction of high molecular weight DNA and purification of the DNA library after tagmentation), both in the form of manual implementation and in the form of an automated protocol.

### Task of the Invention

The invention was based on the task of providing a simple and, above all, fast method for purifying generated libraries for nanopore sequencing and/or, moreover, to generally have a method and a kit that enables the entire sample preparation for nanopore sequencing (extraction of high-molecular DNA and purification of the DNA library). It should be possible to carry out the process manually and also automatically.

### Solution of the Task

Surprisingly, the task can be ideally solved by means of the present invention in accordance with the features of the patent claims.

The invention preferably utilizes the possibility of adsorbing DNA on rough or structured surfaces known, inter alia, from the patent specifications (U.S. Pat. No. 10,851,368 B2; U.S. Pat. No. 10,704,039 B2; U.S. Pat. No. 10,936,540 B2). These patent specifications describe the adsorption of DNA on rough or structured surfaces of nucleic acids released after lysis in the presence of (preferably) ethanol or isopropanol, the subsequent washing of the nucleic acids with ethanol-containing buffers and the final detachment of the nucleic acids by means of a low salt buffer or by means of water. But there is no indication of purifying DNA for the preparation of a library for subsequent nanopore sequencing by means of the Ultra-Long DNA Sequencing Kit (SQK-ULK001; Oxford Nanopore Technologies). The difficulty here is that alcoholic components such as ethanol or isopropanol must not be used, as these components would lead to denaturation of the motor protein coupled to the adaptors. Therefore, a process was needed that allows efficient adsorption of the produced long-chain DNA fragments of the prepared DNA library without the use of ethanol or isopropanol. In the patent specifications cited already above, ethanol or isopropanol is always used for the adsorption of the DNA. Nor must the wash buffers contain these components. In the patent specifications cited above, ethanol or isopropanol is always used for the adsorption of DNA. Nor must the wash buffers contain these components. Surprisingly, a combination of polyethylene glycol (a polyether) with a monohydric or multivalent salt is excellently suited for adsorbing the DNA fragments on known rough or structured surfaces. The adsorbed fragments are then washed by using the method according to the invention without the alcohol-containing wash buffers used up to now. It is shown that a wash buffer using a Tris solution is completely sufficient for this purpose. This wash buffer also has the advantage that alcohol removal is no longer necessary. The process, which is based on the new components according to the invention, is very simple and fast. The same materials known from the listed patent specifications can be used as rough or structured surfaces, Preferably, magnetic plastic materials of different shapes and sizes with rough surfaces are used for manual implementation. For the automated implementation, roughened plastic materials are used which are normally used in commercially available extraction machines for the separation of magnetic beads. These machines are so-called walk-away platforms which use plastic combs for the separation of magnetic beads. A world-renowned system in this regard is the so-called Magnetic Particle Processor “KingFisher” from the company Thermo. A large number of similar machines exist worldwide, all of which use the same principle. No magnetic beads are required for the process according to the invention. The plastic combs are slightly roughened. This then enables direct adsorption of the DNA fragments onto the plastic combs by using the method according to the invention. 1121 For manual purification of the libraries, the reaction mix is mixed with polyethylene glycol and a monohydric or divalent salt in a reaction vessel and brought into contact with a material with a rough or structured surface which is preferably magnetic or paramagnetic. The vessel is carefully inverted several times so that the long-chain DNA fragments are not sheared. The DNA fragments adsorb to the surface. The vessel is placed in a magnetic rack. The rough material is fixed and the supernatant is poured off. A wash buffer without ethanol or isopropanol is then added, preferably a Tris buffer is used, the vessel is left in the magnetic rack and carefully inverted several times. The supernatant is poured off. The washing step is repeated a second time. After the last washing step, ddH2O (double-distilled water) is added. The vessel with the rough materials is then incubated for some time with gentle shaking to allow the bound DNA fragments to dissolve. After this incubation, the DNA library can be stored or used immediately for nanopore sequencing.

Thus, the entire process takes less than an hour and is therefore much faster than the methods previously used. In the automated method using walk-away platforms, which use plastic combs as described above, purification is also carried out by using the reagents described. The DNA fragments are adsorbed onto the plastic combs by slowly moving the comb vertically into the reaction cavity which contains the reaction mixture. Once the adsorption step has been completed, the comb moves into the cavity containing the wash buffer. Washing is carried out by briefly dipping the comb into the buffer. The washing step is repeated. In the final step, the DNA is dissolved in water again. This is done by slowly immersing and surfacing again the comb. Subsequently, the dissolved DNA can be used immediately for nanopore sequencing or is stored in the refrigerator for later use. Automated purification is also completed in less than an hour. The method and means according to the invention thus provide a very simple, efficient and fast way permitting to purify DNA fragments from a generated DNA library for subsequent nanopore sequencing. Said method can be carried out in an automated manner or manually. Surprisingly, a further advantage is revealed when a mixture of polyethylene glycol and monohydric or multivalent salts is used for the adsorption of DNA on rough surfaces.

The method is not only suitable for purifying the DNA library, but also for extracting high molecular weight DNA, which is required for the production of the DNA library. There are commercially available products from NEB and Circulomics for the extraction of high molecular weight DNA. But the purification of high molecular weight DNA is generally labor-intensive and time-consuming. Automated applications are hard to find on a commercial level Another major disadvantage is that the available methods and reagents and/or kits used for the extraction of high molecular weight DNA and the necessary purification of libraries prepared for sequencing differ. There is no kit that allows both procedures to be implemented.

Based on the present invention, it is possible to isolate with a kit high molecular weight DNA from a biological sample and to permit purification of the library. Such a product and/or process does not exist on a worldwide basis. The person skilled in the art would be aware of the advantage of availability of such a solution. Such a kit only requires additional reagents for sample lysis (lysis buffer and proteolytic enzyme) and possibly a wash buffer which may also contain an ethanol-containing component. According to the invention, such a kit for manual extraction of high molecular weight DNA from a biological sample and the subsequent purification of the library would contain the following components:
1. lysis buffer and proteinase K
2. polyethylene glycol solution
3. magnesium chloride solution (as a multivalent salt)
4. optionally an ethanol based wash buffer
5. Tris solution as a wash buffer without alcoholic components
6. material with rough surface made from plastic which is magnetic (granules or beads)
7. ddH₂O

The extraction procedure is briefly outlined below:

### Extraction of High Molecular Weight DNA

1. Lysis of the initial sample (cells, tissue, bacteria, etc.) by adding a lysis buffer and proteinase K,
2, After the initial sample has been lysed, a defined amount of polyethylene glycol and magnesium chloride is added and the rough material is added.
3. Careful inverting of the reaction vessel several times.
4. Placing of the vessel in a magnetic rack. Fixing the rough material with the adsorbed DNA and pouring off the supernatant.
5. Addition of a wash buffer containing ethanol and inverting the vessel three times and pouring off the supernatant. Repetition of the step.
6. Addition of a Tris solution and inverting the vessel three times and pouring off the supernatant.
7 Addition of water and dissolving the DNA by incubation in a thermoshaker at 300 rpm and 37° C. for approx. 30 min.

After this time, the high molecular weight DNA can be used directly for the production of a DNA library for nanopore sequencing. It is of course also possible to subsequently store the DNA properly and use it at a later date.

For nanopore sequencing by means of the Ultra-Long DNA Sequencing Kit SQK-ULK001 (Oxford Nanopore Technologies), a defined amount of DNA is used to prepare the DNA library. After this step is completed, the library is purified by using the method and kit according to the invention as follows:
1. Addition of a defined quantity of polyethylene glycol and magnesium chloride as well as addition of the rough material to the reaction mixture with the produced library.
2. Careful inverting the reaction vessel several times.
3. Placing of the vessel in a magnetic rack. Fixing the rough material with the adsorbed DNA and pouring off the supernatant.
4. Addition of a Tris solution for washing the adsorbed DNA and inverting the vessel three times and pouring off the supernatant.
5. Addition of water and dissolving the DNA by incubation in a thermoshaker at 300 rpm and 37° C. for approx. 30 min.

The DNA can now be applied directly to the flow cell for sequencing, but can also be stored and used at a later date.

Similarly, the combined extraction of high-molecular weight DNA and the subsequent purification of the library can be automated. This is done by means of the previously described use of walk-away platforms using rough plastic combs. The process is simple to perform and very fast. In addition, very long fragments can be sequenced by using nanopore sequencing. This is a very important quality feature.

The invention is described below with reference to embodiments.

### EMBODIMENTS

### Embodiment 1

Manual Extraction of High Molecular Weight DNA from Blood for Subsequent Nanopore Sequencing by Means of the Ultra-Long DNA Sequencing Kit SQK-ULK001 (Oxford Nanopore Technologies) Including Purification of the Generated DNA Library

3 ml of a whole blood sample was used. After lysis of the erythrocytes, the resulting cell pellet of the nucleated blood cells was resuspended with 130 μl of 1×PBS buffer and transferred to a 2.0 ml reaction vessel. Then 200 μl of a lysis buffer (Lysis Solution CBV; IST Innuscreen GmbH) and 30 μl proteinase K were added. Cell lysis was carried out at 55° C. in a thermomixer for 15 min. After lysis has been made, 3 paramagnetic plastic beads with a roughened surface (diameter 3 mm; polypropylene) were added to the preparation. Then 200 μl of a 40% polyethylene glycol solution (PEG solution) and 40 μl of magnesium chloride (400 mM) were added. The vessel was then carefully inverted by 180° 30 times. In this step, the high molecular weight DNA adsorbs to the rough surface of the paramagnetic beads. The vessel was then placed in a magnetic rack to fix the beads and the lysis mixture was poured out. A washing buffer (Washing Solution MS; IST Innuscreen GmbH) was then added. The vessel was left on the magnetic rack and inverted 3 times and the wash buffer was poured off. The washing step was repeated once more. Then 800 μl of a Tris solution (100 mM Tris-HCl; pH 8) was added. The vessel was inverted twice and the Tris solution, was poured off. Due to this final washing step with a non ethanol containing solution, a final drying step for complete ethanol removal is no longer necessary. The DNA on the beads was released after addition of 200 μl ddH₂O. For this purpose, the vessel was incubated at 300 rpm and a temperature of 37° C. for approx. 30 min. The high molecular weight DNA was transferred to a new vessel by means of a wide-bore pipette tip and carefully pipetted up and down several times in the process. The DNA was measured spectrophotometrically and a quantity of 45 μg DNA in a volume of 400 μl was used for further nanopore sequencing by using the Ultra-Long DNA Sequencing Kit SQK-ULK001 (Oxford Nanopore Technologies). Once the DNA library has been prepared, it is necessary to remove excess adaptors. This was subsequently carried out by using the method according to the invention. For this purpose, the preparation was again mixed with a PEG solution (40%) and a magnesium chloride solution (400 mM) as well as with roughened paramagnetic plastic beads. The vessel was then carefully inverted by 180° 30 times to adsorb the high molecular weight DNA onto the rough surface of the paramagnetic beads. Subsequently, the vessel was placed in a magnetic rack to fix the beads and the reaction mixture was poured off. Then 800 μl of a Tris solution (100 mM Tris-HCl; pH 8) was added. The vessel was inverted twice and the Tris solution was poured off. The washing step was repeated once more. When purifying the DNA library, it was important that no alcoholic components are used as wash buffers or binding buffers, as this would lead to destruction of the library. The DNA on the beads was dissolved after adding 225 μl ddH2O. For this purpose, the vessel was incubated for approx. 45 min at 300 rpm and a temperature of 37° C. After DNA extraction, preparation of the DNA library and purification of the DNA library, the sample was transferred to the Minion device (Oxford Nanopore Technologies) for long-read sequencing. By means of this technology, it could be shown that very high read lengths (read lengths up to >300,000 base pairs) can be achieved by means of the method according to the invention with the combination of extraction of high-molecular DNA and purification of the DNA library. The entire procedure is completed in approx. 2 hours which allows sequencing to be started on the same working day. In addition, the procedure is very easy to perform.

### Embodiment 2: Automated Extraction of High Molecular Weight DNA from Blood for Subsequent Nanopore Sequencing Using the Ultra-Long DNA Sequencing Kit SQK-UILK 001 (Oxford Nanopore Technologies) Including Purification of the Generated DNA Library by Using a Walk-Away Platform (KingFisher Flex; Thermo)

3 ml of a whole blood sample was used. After lysis of the erythrocytes, the resulting cell pellet of the nucleated blood cells was resuspended with 130 μl 1×PBS buffer and transferred to a cavity of a 96-well deep well plate for processing the automated protocol on the KingFisher Flex device. In the automated method using walk-away platforms (as here using the KingFisher Flex device), the high molecular weight DNA is adsorbed onto the plastic combs described above which are used by these device systems. The extraction was also carried out by using the reagents described for the manual process. After automated sample lysis on the device has been made, the DNA fragments were adsorbed onto the plastic combs by a slow vertical movement of the comb in the reaction cavity in which the reaction mixture is located. After the adsorption step of the high molecular weight DNA has been completed, the comb moves successively into the deep well plate cavities with the wash buffers, wherein during extraction of the high molecular weight DNA washing occurs with an ethanol containing wash buffer. The final washing step is then carried out again with a Tris solution. The high molecular weight DNA was transferred from the cavity of the deep-well plate into a new vessel by using a wide-bore pipette tip which is carefully pipetted up and down several times in the process. The DNA was measured spectrophotometrically and a quantity of 45 μg DNA in a volume of 400 μl was used for further nanopore sequencing by using the Ultra-Long DNA Sequencing Kit SQK-ULK001 (Oxford Nanopore Technologies). Once the DNA library had been prepared, the automated purification of the DNA library was carried out by using the reagents already listed in embodiment 1 in order to remove the excess adaptors. In the automated purification of the DNA library, only the Tris solution is used for washing. Washing is performed by briefly dipping the comb into the respective buffer. In the final step, the DNA is then dissolved in water again. This is carried out by slowly dipping the comb in and out of the water. After automated extraction of high-molecular weight DNA, preparation of the DNA library and automated purification of the DNA library, the sample is transferred to the Minion device (Oxford Nanopore Technologies) for long-read sequencing. By means of this technology it could be shown that very high read lengths (read lengths of up to >300,000 base pairs) can be achieved by using the method according to the invention of combining automated extraction of high-molecular DNA and purification of the DNA library. The entire procedure is completed in approx. 2 hours which allows sequencing to be started still on the same working day. Furthermore, the procedure is very easy to perform.

### Definitions

### Monohydric Alcohol:

Alcohols with only one OH group such as ethanol or isopropanol

### Polyether:

### Long-Chain Compounds of the Type

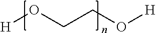

are called polyethers (also polyalkylene glycols, polyether polyols, polyalkylene oxides). Examples of this group of polymeric ethers are polyethylene glycol and polypropylene glycol. PEG 6000 or PEG 8000 means that n=6000 or 8000 respectively.

### TRIS:

Tris(hydroxymethyl)aminomethane (abbreviation TRIS or THAM), also known as tromethamine, trometamol (INN) and TRIS buffer. Chemically, it is a primary amine with three alcoholic hydroxy groups. Preferably its salt (hydrochloride) is used with a pH value of 7-9, preferably pH=8.

### High Molecular Weight DNA:

DNA with more than or equal to 100,000 bp.

1: A method for extracting high-molecular weight DNA from a biological sample and/or purifying a DNA library, the method comprising:
after optionally lysing the biological sample and/or preparing the DNA library, contacting the preparation with a combination of a polyether and at least one salt and in the absence of monohydric alcohols, then
binding the DNA to a solid phase, and then
purifying the DNA without using monohydric alcohols.

2: The method according to claim 1, wherein the polyether is polyethylene glycol (PEG).

3: The method according to claim 1, wherein MgCl2 is used as the salt.

4: The method according to claim 1, wherein the poly ether and salt are present together in a buffer in corresponding concentrations.

5: The method according to claim 1, wherein the solid phase is a material with a rough or structured surface.

6: The method according to claim 1, wherein purifying the DNA comprises purifying the DNA with a wash buffer without monohydric alcohol and wherein the wash buffer is an amino alcohol.

7. A method for extraction of high molecular weight DNA from a biological sample, the method comprising:
lysing the biological sample,
adding polyethylene glycol and magnesium chloride and a rough material,
fixing the rough material with the adsorbed DNA and pouring off supernatant,
adding a wash buffer, and
adding water and dissolving the DNA

8: A method for purifying a DNA library, the method comprising:
adding polyethylene glycol and magnesium chloride and a rough material to a reaction mixture with the generated library,
fixing the rough material with the adsorbed DNA and pouring off a supernatant,
adding, a wash buffer without monohydric alcohol, and
adding water and dissolving the DNA.

9: The method according to claim 1,
wherein the method is automated,
wherein the method comprises the lysing of the biological sample and/or the preparing of the DNA library in a walk-away extraction device which uses plastic rods, wherein the plastic rods have a rough or structured surface to which the DNA adsorbs by adding polyethylene glycol and magnesium chloride by vertical movement of plastic combs within the sample, and wherein the plastic combs move according to a walk-away principle into vessels with a wash buffer and finally into an aqueous solution.

10: A kit for manually performing the method according to claim 1, the kit comprising:
a lysis buffer,
a polyethylene glycol solution,
a magnesium chloride solution,
a Tris solution as a wash buffer,
a rough surface plastic material which is magnetic and which comprises granules or beads, and
ddH2O.

11: A nanopore sequencing method, comprising:
extracting high-molecular weight DNA from a biological sample and/or purifying a DNA library by the method according to claim 1.

12: The method according to claim 1, wherein the polyether is PEG 6000 or PEG 8000.

13: The method according to claim 1, wherein MgCl2 is used as the salt in a concentration of between 1 and 100 mM.

14: The method according to claim 1, wherein purifying the DNA comprises purifying the DNA with a wash buffer without monohydric alcohol and wherein the wash buffer is TRIS.
